(19) United States
(12) Patent Application Publication
(10) Pub. No.: US 2012/0253364 A1
(43) Pub. Date: Oct. 4, 2012

(54) Surgical Instrument Handle and Grip

(76) Inventor: James C. Perin, (US)

(21) Appl. No.: 13/220,745

(22) Filed: Aug. 30, 2011

Related U.S. Application Data
(60) Provisional application No. 61/468,104, filed on Mar. 28, 2011.

(51) Int. Cl. A61B 17/28; A61B 17/04; A61B 17/00

(52) U.S. Cl. 606/148; 606/1; 606/205

(57) ABSTRACT

The Ergonomic Surgical Instrument Handle described herein is designed to efficiently grasp, maneuver and clamp tissues, objects and vessels within the surgical site. Surgical Instruments with the improved handle are ergonomically designed to be in line with the surgeon's wrist and hand as they relate to the surgical site. This novel design makes it easier to maneuver the instrument within the surgical site without awkward angles to the wrist and hand which reduces muscle fatigue and cramping.

### CROSS REFERENCE TO RELATED PATENT APPLICATIONS

This application claims the benefit of priority of U.S. provisional patent application Ser. No. 61/468,104, filed Mar. 28, 2011, said application being relied upon and incorporated herein by reference.

### BACKGROUND OF THE INVENTION

1. Field of Invention

This invention relates to handles for surgical instruments. In particular, this invention relates to an improved handle for hand held surgical instruments used in the surgical setting and healthcare industry. The surgical instruments herein described have at least one articulating member and the ability to lock the instrument as well as open and close the tips of the instrument.

2. Background Art

Surgery is a learned skill requiring many years of training to develop an understanding of surgical procedures, disease processes and healing that far exceed the basic medical principles. The surgeon must develop hand-to-eye coordination and acquire skills utilizing a variety of highly specialized surgical instruments. The surgical instruments and tools are an extension of the surgeon's hand. The surgeon's ability to perform the surgical procedures with instruments and tools designed to benefit skill is paramount to the successful outcome for the patient. To enhance the surgical performance to better serve the patient means developing instrument handles which are responsive, sensitive and ergonomically designed to benefit the natural motions of the human hand.

The handles of currently available surgical instruments (forceps, hemostats, clamps, needle drivers, etc.) are usually configured with loops or rings for the surgeon to place their fingers through. Because the standard grip used in surgical procedure and taught to surgeons is the palm grip (placing the surgical instrument within the surgeon's hand and grasping one handle with the distal phalanges and using the base or tip of the thumb to open and close the instrument) the loops on the handle are more a nuisance than a help. The loops also limit the surgeon to using only the palm grip since the loops get in the way of other surgical grips like the pencil grip. In their training surgeons are taught not to use the loops of the surgical instruments because of the blood, tissue and bodily floods on the surgical glove can cause the fingers to get stuck in the loops and tear tissue if the surgeon pulls the hand away without understanding they are stuck in the loops of the instrument.

The loops (or rings) on the handle of current surgical instruments also require more space and thus clutter up the surgical site. This clutter decreases the visual space the surgeon has for viewing the surgical site. The size of the loops also causes problems with manipulating, clamping or grasping tissue or objects in a small, tight space. An example is when a surgeon is suturing with a surgical needle driver using a palm grip. The surgeon must rotate the surgical needle driver to throw a stitch and suture the layer of tissue closed. Because of the size of the loops, the area the surgeon must work in is significantly large or the surgical needle driver will catch on tissue or not be able to turn at all.

The present invention satisfies the need in the art for a more sensitive, useful and ergonomic handle by providing a surgical instrument handle that is designed to work with the surgical grips taught to surgeons. The surgical instrument handle herein described is a natural gripping mechanism and motion that exists between the wrist and hand of the surgeon. The present invention additionally provides a means for locking the handle in a variety of closed positions. The invention satisfies the need for a surgical handle that is simpler to manufacture and assemble than the prior art.

### SUMMARY OF THE INVENTION

The present invention provides a surgical instrument with an improved instrument handle consisting of a cylinder and grooved surface for better gripping. The instrument is designed to be held in a palm grip, pencil grip or modified grip positions; all of which are natural gripping relationships between the surgeon's wrist and hand.

The cylinder (or barrel shaped) handle of the surgical instrument along with the grooved grip of the invention provides great versatility in gripping positions and gripping power. In a palm grip, one handle is stabilized by wrapping the distal phalanges around the cylinder and using the base or tip of the thumb to manipulate the other handle open or closed. In a pencil grip type of position, the instrument handle is stabilized by the base of the second phalange and held between the third and fourth phalanges while manipulating the instrument open and closed with the tip of the thumb and second phalange. The ergonomic design permits the surgeon to transfer between grips and positioning of the instrument in direct relationship from the wrist and hand to the surgical tool with precision, ease and delicacy.

The invention further provides an ergonomic grooved grip along the length of the cylinder on the handle for superior grip and traction of the surgeon's hand and phalanges. Furthermore, the instrument may be rotatable through a variety of grips. The instrument may be equipped with a means for locking the handle in a variety of closed positions.

Additionally, the cylindrical handle of the invention is designed for easy manufacture and assembly. These and other features of the present invention will become apparent in light of the specification as a whole.

### DESCRIPTION OF THE DRAWINGS

The invention will be more clearly understood from the following description of some embodiments thereof given by way of example only with reference to the accompanying drawings in which:

FIG. 1 is a plan view in the closed position of a surgical instrument (ie, surgical forceps) according to the present invention;

FIG. 2 is a plan view of the surgical instrument in the open position;

FIG. 3 is a plan view of three surgical instruments demonstrating the position of the surgical handle in relation to the other as well as the variable length of the handle;

FIG. 4 is an exemplary representation of the surgical instrument within the surgical hand using a palm grip to hold and manipulate the instrument closed and opened, respectively;

FIG. 5 is an exemplary representation of the surgical instrument within the surgical hand using a pencil grip to hold and manipulate the instrument.

FIG. 6 is a plan view of closed surgical instrument with adjustable joints at the base of the handle.

FIG. 7 is a plan view of the surgical handle that is hollow for storage of medicine, suture needle and thread, etc. with a screw cap for opening and closing the storage area.

FIG. 8 is a plan view of removable grips;

FIG. 9 is a plan view of a grip with the ability to extend its length;

FIG. 10 is an illustrative representation of a prior art surgical instrument handle.

The following description will be set forth in connection with a preferred embodiment, however, it will be understood that there is no intent to limit the claimed invention to the embodiments described. On the contrary, the intent is to cover all alternatives, modifications, and equivalents as may be included within the spirit and scope of the appended claims.

### DESCRIPTION OF THE INVENTION

For a general understanding of the disclosed embodiments, reference is made to the drawings. In the drawings, like reference numerals have been used throughout to designate identical elements. In describing the embodiments, the following term(s) have been used in the description. The term “surgical instrument” or “instrument” is intended to encompass not only those surgical instruments intended for the grasping and manipulation of tissue, objects or vessels within the surgical site or wound, but to similarly constructed instruments suitable for grasping other surgical implements, suture needles and the like (ie surgical forceps, hemostats, needle drivers, clamps and retractors).

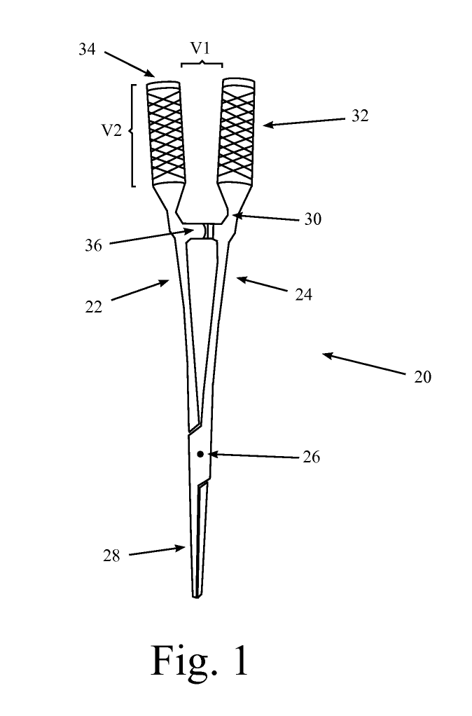

Referring now to FIG. 1, there is depicted a surgical instrument with the surgical handle herein described to overcome the aforementioned problems of fatigue, cramping, unusual pressure points and awkward angles of the surgeon's hand and wrist in relation to the surgical site or wound. It will be appreciated that such a handle would find practical application with surgical instruments in which the surgeon uses a palm grip, pencil grip or other grip in which the loops of conventional surgical handles are not used. Depicted in FIG. 1 is a surgical instrument such as a surgical forceps 20. It can be seen that the instrument comprises a pair of shanks 22 and 24, which are hinged or pivotally connected to each other at a joint 26. Each of the shanks 22 and 24, are formed of a similar shape, but are somewhat different so the shanks do not interfere with one another during use.

Continuing with FIG. 1, the instrument is divided into three sections, the lower or first end of each shank, there are opposing surfaces that form a jaw or blade surface 28 therebetween. Although not depicted in particular detail, it will be appreciated that the jaw, and the associated opposing surfaces, may be of various shapes and configurations. Although the embodiments depicted in FIG. 1, illustrate planar opposing surfaces, other shapes are contemplated and included within the scope of this disclosure. It will be appreciated that for the purposes of holding, grasping or manipulating a tissue, object, implant or suture needle, the surfaces should be mating surfaces (meaning the jaws should close to grasp, hold or manipulate without interference). The midsection of the pair of shanks 22 and 24 allows for force applied to the surgical handle 32 and 34 to flex the shanks for locking and unlocking of the ratchet lock 36. The midsection of the pair of shanks can be thicker in diameter to make the shanks a less flexible instrument and vary in length for different instruments. On the opposite end of each shank 22 and 24, is a barrel shaped handle with a knurled or striated finish for gripping. The barrel shape handle 32 and 34 at the upper end of the pair of shanks 22 and 24 facilitate the manipulation of the instrument without using the finger or thumb. Such a configuration provides greater strength and less fatigue when using the surgical instrument for surgical procedures. Illustrated is the distance between the pair of handles V1 and the length of the barrel shaped handle V2.

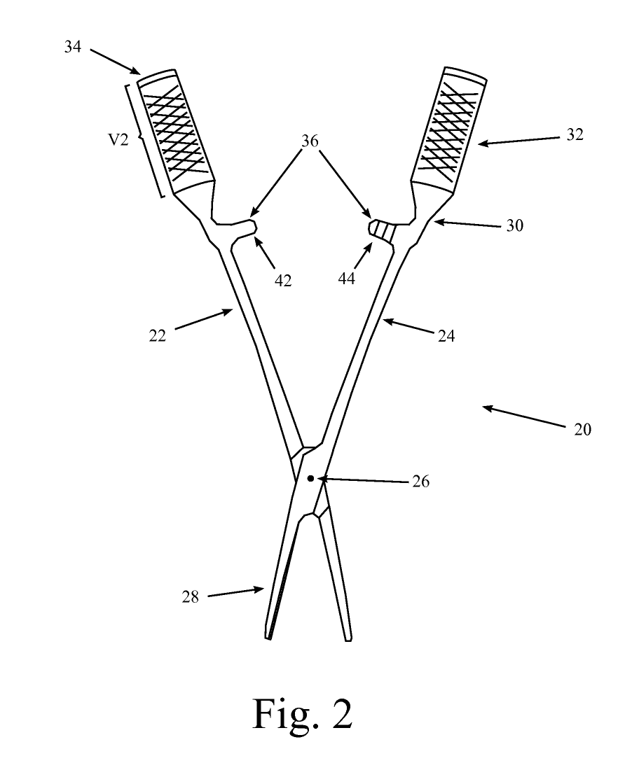

Referring to FIG. 2, there is shown the instrument of FIG. 1, in an open configuration. The jaws are opened to receive tissue or implants for holding, grasping or manipulating or the like therein. The upper section of each shank has associated therewith a locking mechanism 36. As depicted in FIG. 1, mechanism 36 is a ratchet mechanism, wherein each shank includes a tab 42 extending therefrom, where at least one side has a series of angled grooves or cuts 44, and the grooves may be suitably mated with grooves on a tab of the opposing shank to lock the shanks.

Such mechanisms are well known to those familiar with surgical instruments. Although a ratchet is shown in the figures, it will be appreciated that other mechanisms may be employed to bias, hold or lock the instrument 20 in a generally closed position—where the opposing surfaces at the opposite ends of the shanks are urged to contact on another (example to hold a tissue therebetween). There are alternative configurations considered but again such mechanisms are known for use in medical instruments as well as other hand-operated tools.

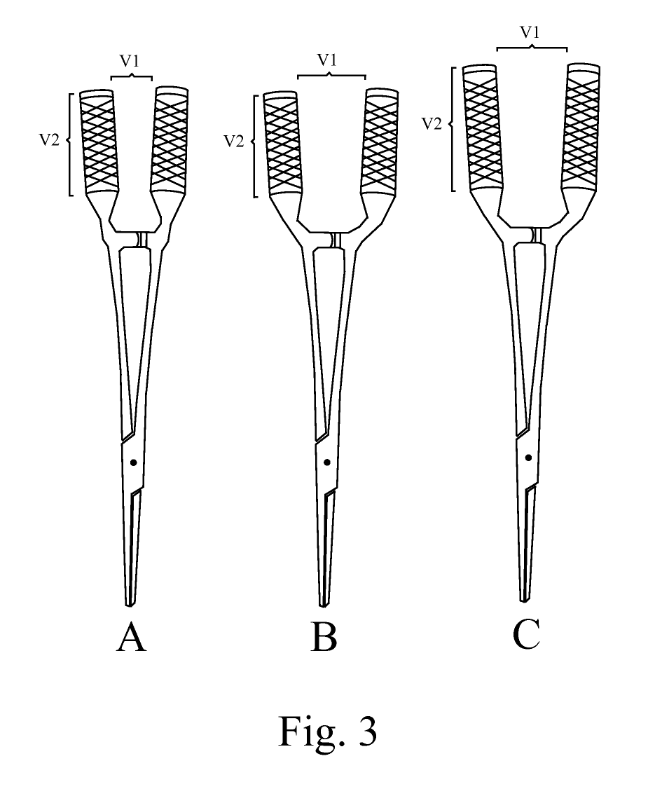

Referring now to FIG. 3, the distance between the pair of handles V1 is determined by closing and locking the instrument to the last ratchet. This distance V1, can be made narrower or wider depending on the surgical instrument and the surgeon's preference of grip. For a surgeon who prefers the palm grip for said instrument, the handle would have a wider distance between the pair of handles for leverage as seen in FIG. 3B&C. For a surgeon who prefers a pencil grip, the distance between the pair of handles V1 would be narrower as seen in FIG. 3A. For surgeons with different size hands, the length of the handle V2 can be made longer (FIG. 3C) or shorter (FIG. 3A&B) depending on the preference of the surgeon.

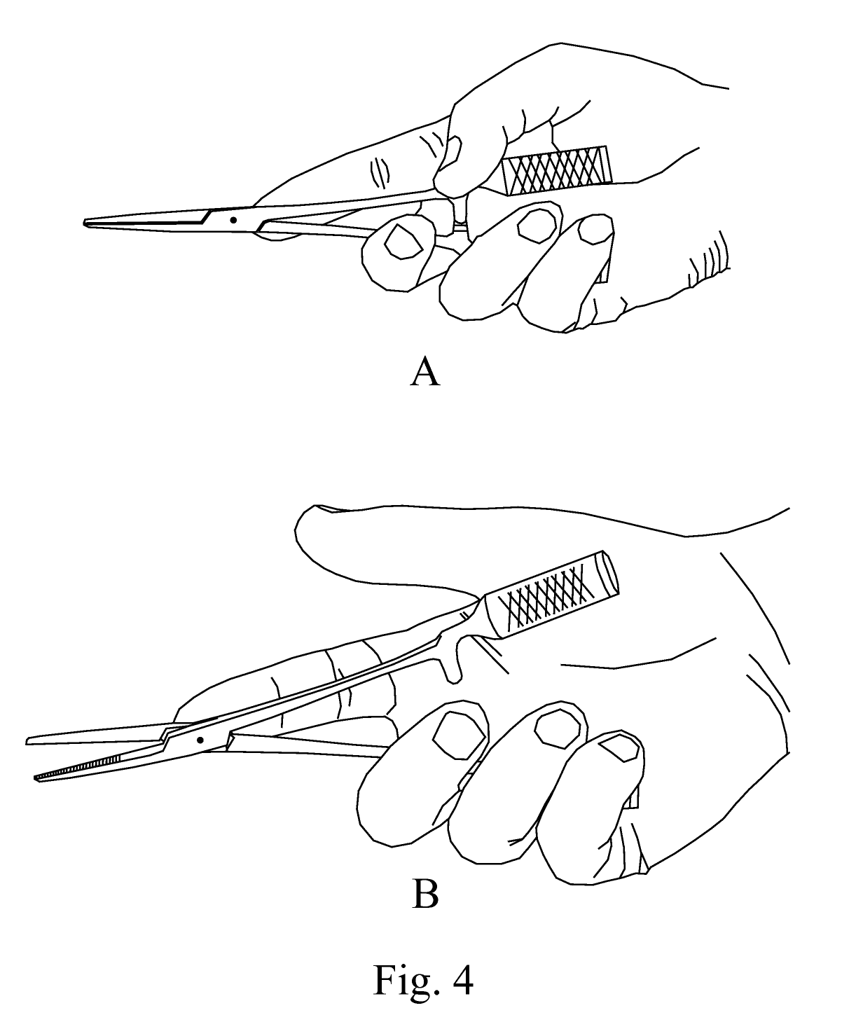

Referring now to FIG. 4, the surgical instrument is held within the surgical hand in a palm grip. The base or tip of the thumb is placed against the handle 32 to manipulate the instrument open and closed. The distal phalanges (2 thru 5) are used to stabilize the instrument by wrapping around the other handle 34 as seen in FIG. 4A. As the thumb is extended, the handle 32 moves away from the handle 34 to open the instrument as seen in FIG. 4B.

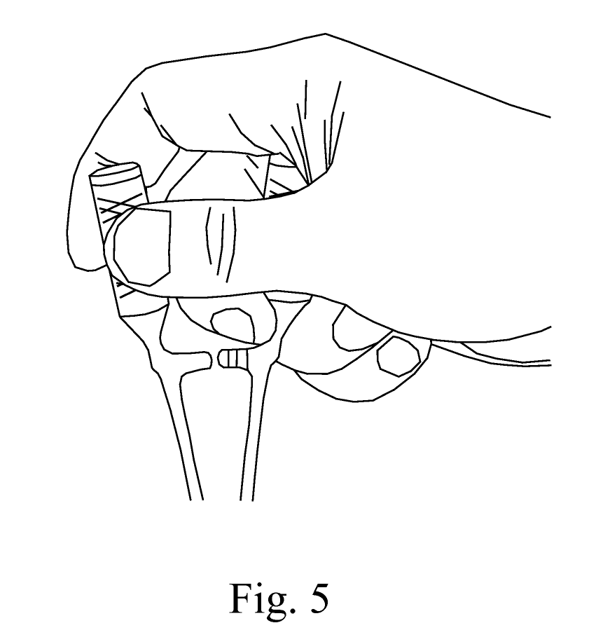

Referring to FIG. 5, a pencil grip is demonstrated as the surgical hand holds the handle 32 between the tip of the second phalange and the tip of the thumb. The other handle 34 is held in the crux of the base of the thumb and second phalange or against the first metacarpal joint of the second phalange to stabilize the instrument. The third phalange is placed in the groove below the handle 34 while the fourth phalange is placed on the opposite side of the groove to stabilize the instrument further.

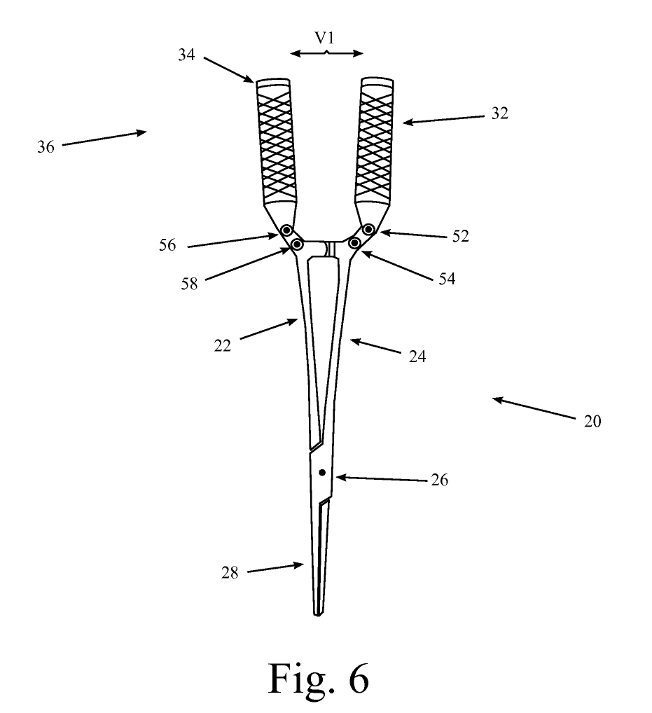

Referring now to FIG. 6, which is an alternative configuration where the pair of handles is designed to have a joint mechanism 52, 54, 56, 58 so the distance between the pair of handles V1 can be set by the surgeon or surgical staff to accommodate the surgeon's preference. The adjustable joints 52, 54, 56, 58 are designed to be unlocked with a turn of a lever which allows the joint to rotate making the handles 32 and 34 move closer or further apart. Once the distance and angle of the handles 32 and 34 is set the surgeon can lock the handle in place by turning the lever back to its starting position.

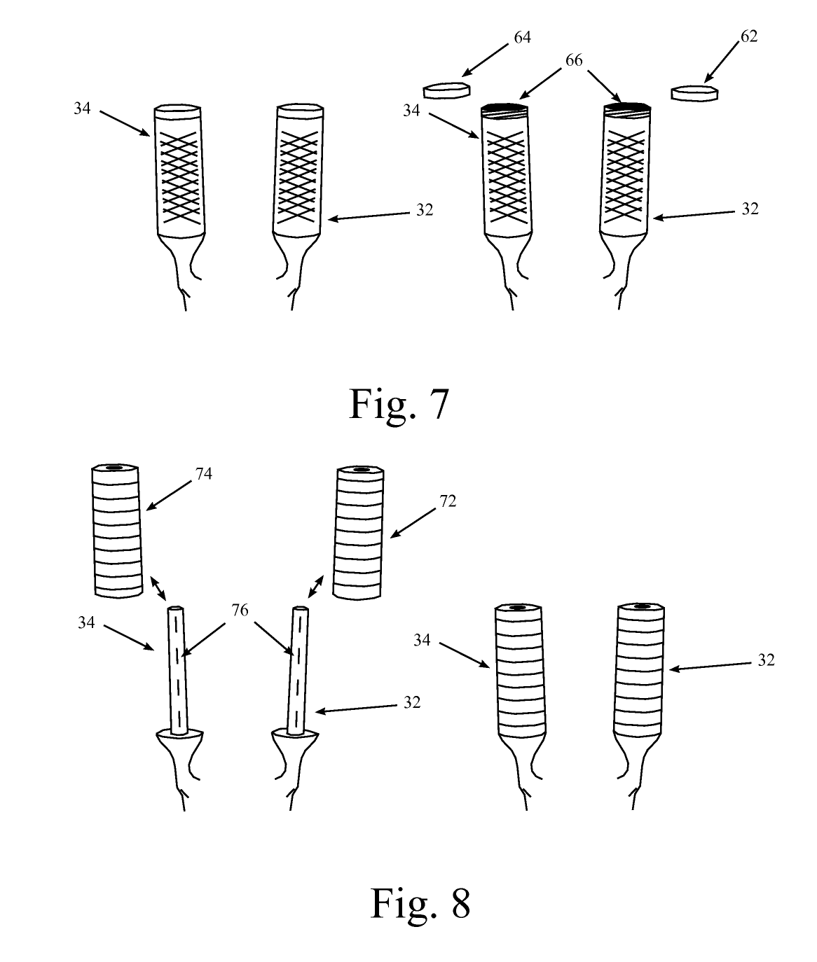

Referring now to FIG. 7, the pair of handles 32 and 34 is made with a hollow center 66. A screw top 62 and 64 is used to open and close the handle where items can be stored (ie suture needle and thread or medicinal items as needed).

Referring now to FIG. 8, the pair of handles 32 and 34 of the surgical instrument are posts with grooved slots 76 for placing a single use gel grip 72 and 74 over the metal posts. The surgical nurse or staff can place the sterilized gel grips on the instrument at the beginning of the surgery and remove them when the surgery is completed for waste. The center of the gel grips is a hard plastic with ridges that fit into the slots of the surgical post so the gel grips doesn't turn or slip when in use.

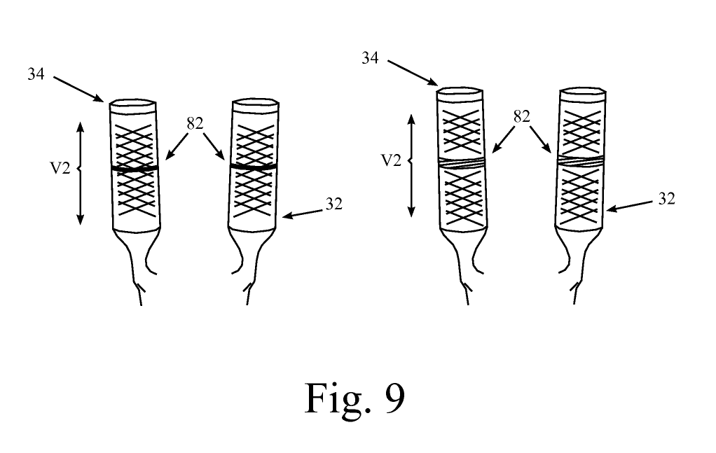

Referring now to FIG. 9, the pair of handles 32 and 34 has a turning, thread mechanism 82 in the middle which allows the surgeon to rotate the handle against the instrument. As the handle is rotated, the length of the handle can be longer or shorter depending on the direction of rotation thus allowing the surgeon to set the length of the handle to their preference.

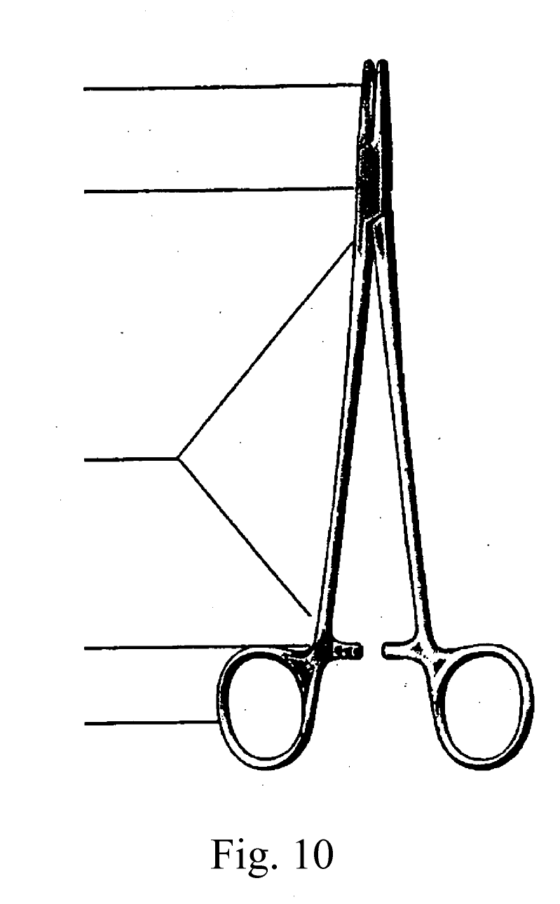

Referring to FIG. 10, this is prior art demonstrating the loops or rings that have been replaced on surgical instruments.

What is claimed is:

1. A surgical instrument with an ergonomic handle comprising:
the handle assembly is an extension of both shafts of the surgical instrument handle;
the handle of the shafts can vary in length, diameter and distance apart for different surgical procedures, specialties or needs;
the handle of cylinder, barrel or elliptical shape has grooves and/or ridges for improved grip;
the handle can be hollow for storage of materials;
the handle can have articulating joints at the base which allow for changing the distance one handle is in relation to the other handle.
a handle assembly comprising two similar cylinder, barrel or elliptical shapes to extend and elongate the shaft of the surgical instrument;

2. The ergonomic handle of claim 1 wherein the handle is used for multiple surgical grips which keep the surgeons hand and wrist at ergonomic angles to the surgical site.

3. The ergonomic handle of claim 2 wherein the surgical hand can use the distal phalanges to stabilize one handle and the base or tip of the thumb to manipulate the surgical instrument open and closed.

4. The ergonomic handle of claim 2 wherein the base of the second phalange along with the tip of the third and fourth phalange stabilize one handle while the tip of the thumb and second phalange manipulate the surgical instrument open and closed.

5. The ergonomic handle of claim 1 wherein a single handle comprises a stabilizing means and the second handle comprise an opening and closing means.

6. The ergonomic handle of claim 1 wherein the handle of a surgical instrument that comprises a clamping mechanism.

7. The ergonomic handle of claim 1 wherein the handle of a surgical instrument that comprise a needle drivers.

8. The ergonomic handle of claim 1 wherein the handle of a surgical instrument that comprise a manipulating mechanism.

9. The ergonomic handle of claim 1 wherein the handle of a surgical instrument that comprise a grasping mechanism.
